(19) United States
(12) Patent Application Publication
(10) Pub. No.: US 2024/0099568 A1
(43) Pub. Date: Mar. 28, 2024

(54) ENDOSCOPE AND IMAGE PICKUP UNIT

(71) Applicant: OLYMPUS CORPORATION, Tokyo (JP)

(72) Inventor: Yuichi WATAYA, Tokyo (JP)

(73) Assignee: OLYMPUS CORPORATION, Tokyo (JP)

(21) Appl. No.: 18/526,465

(22) Filed: Dec. 1, 2023

Related U.S. Application Data
(63) Continuation of application No. PCT/JP2021/022397, filed on Jun. 11, 2021.

(51) Int. Cl. A61B 1/05; A61B 1/00; A61B 1/045

(52) U.S. Cl. CPC A61B 1/05; A61B 1/0016; A61B 1/045

(57) ABSTRACT

An image pickup unit includes a rear group lens barrel which holds the rear group lens, a moving lens barrel which holds the moving lens and which is movable to advance and retreat inside the rear group lens barrel along an optical axis of the observation optical system, an actuator which includes a guide tube and a head member and which is configured to transmit motive power to the moving lens barrel by advance and retreat movement of the head member with respect to the guide tube, and a second stopper member which is positioned and fixed to the rear group lens barrel and which defines a retreat position in a direction of the optical axis of the moving lens barrel, in which the second stopper member also serves as a holding member which holds a distal end side of the guide tube.

### CROSS REFERENCE TO RELATED APPLICATION

This application is a continuation application of PCT/JP2021/022397 filed on Jun. 11, 2021, the entire contents of which are incorporated herein by this reference.

### BACKGROUND OF THE INVENTION

### 1. Field of the Invention

The present invention relates to an endoscope which can cause an optical characteristic of an observation optical system to be changed, and an image pickup unit used in the endoscope.

### 2. Description of the Related Art

As is well-known, an endoscope is widely used for observation, treatment, or the like of an inside of a living body (inside a body cavity), or inspection, servicing, or the like in industrial plant equipment. In recent years, in some endoscopes of this type, an image pickup unit which can change a focal distance (optical characteristic) for a zooming function to perform a focus adjustment of a shooting image or a magnification adjustment such as wide/tele has been used.

In particular, in an endoscope that requires miniaturization, an image pickup unit of a focus switching system in which a focused state is achieved when a moving lens unit (moving lens barrel) provided in an observation optical system is at a predetermined advance position and a predetermined retreat position is widely adopted.

In an image pickup unit of this type, as a technology for setting the retreat position to be at an appropriate position, for example, a technology disclosed in International Publication No. 2016/147468 is known. According to the technology of International Publication No. 2016/147468, a stopper member which allows insertion of a push rod protruding from a holding bar (holding member) is positioned and fixed in a state of being abutted against an outer circumferential surface of a rear group lens barrel. In this manner, movement toward a retreat side along an optical axis of the moving lens barrel by abutment between the stopper member and an operation bar of a moving lens barrel is restricted, and the moving lens can be held at a position where a second focal distance is realized.

### SUMMARY OF THE INVENTION

An endoscope according to an aspect of the present invention includes an observation optical system which is provided in a distal end portion of an insertion section inserted into an inside of a subject and which includes a fixed lens and a moving lens, a fixed barrel which holds the fixed lens, a moving barrel which holds the moving lens and which is movable to advance and retreat inside the fixed barrel along an optical axis of the observation optical system, an actuator which includes a movement section main body and a motive power transmission member and which is configured to transmit motive power to the moving barrel by advance and retreat movement of the motive power transmission member with respect to the movement section main body, and a stopper member which is positioned and fixed to the fixed barrel and which defines a retreat position in a direction of the optical axis of the moving barrel, in which the stopper member also serves as a holding member which holds a distal end side of the movement section main body.

An image pickup unit according to an aspect of the present invention includes an observation optical system which includes a fixed lens and a moving lens, a fixed barrel which holds the fixed lens, a moving barrel which holds the moving lens and which is movable to advance and retreat inside the fixed barrel along an optical axis of the observation optical system, an actuator which includes a movement section main body and a motive power transmission member and which is configured to transmit motive power to the moving barrel by advance and retreat movement of the motive power transmission member with respect to the movement section main body, and a stopper member which is positioned and fixed to the fixed barrel and which defines a retreat position in a direction of the optical axis of the moving barrel, in which the stopper member also serves as a holding member which holds a distal end side of the movement section main body.

### BRIEF DESCRIPTION OF THE DRAWINGS

FIG. 1 is an overall configuration diagram of an endoscope system;

FIG. 2 is a cross sectional view illustrating an internal configuration of a distal end portion and a bending portion;

FIG. 3 is a cross sectional view illustrating a configuration of an image pickup unit in a state where a moving lens unit moves to an advance position:

FIG. 4 is a cross sectional view illustrating a configuration of the image pickup unit in a state where the moving lens unit moves to a retreat position:

FIG. 5 is an exploded perspective view of an observation optical system unit and a solid state image pickup device holding barrel;

FIG. 6 is a perspective view of the observation optical system unit in a state where the moving lens unit moves to the advance position:

FIG. 7 is a perspective view of the observation optical system unit in a state where the moving lens unit moves to the retreat position:

FIG. 8 is a cross sectional view illustrating a configuration of the observation optical system unit along a line VIII-VIII in FIG. 3:

FIG. 9 is a cross sectional view illustrating a binding structure of an image pickup cable and an actuator; and

FIG. 10 is a cross sectional view illustrating a modification of the binding structure of the image pickup cable and the actuator.

### DETAILED DESCRIPTION OF THE PREFERRED EMBODIMENTS

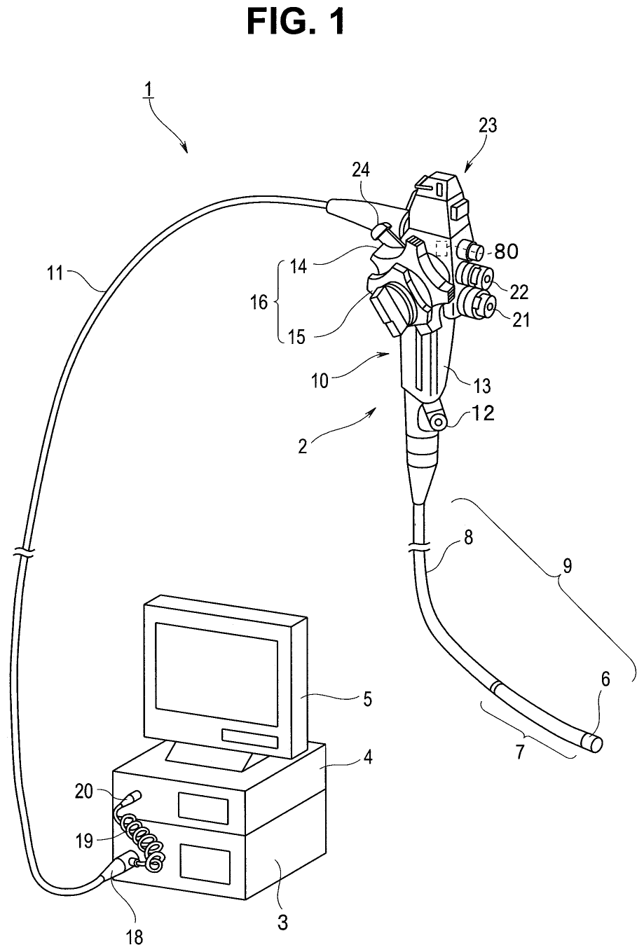

Hereinafter, embodiments of the present invention will be described with reference to the drawings. The drawings are related to an embodiment of the present invention, and FIG. 1 is an overall configuration diagram of an endoscope system. An endoscope system 1 illustrated in FIG. 1 is configured in which an endoscope 2, a light source apparatus 3, a processor 4, and a monitor 5 are electrically connected to one another.

The endoscope 2 is configured by including an insertion section 9, an operation section 10 connected to a proximal end side of the insertion section 9, and a universal cord 11 extending from the operation section 10.

Herein, a scope connector 18 used for connection to the light source apparatus 3 is provided on an extending end of the universal cord 11. A coiled scope cable 19 extends from the scope connector 18. Furthermore, an electrical connector 20 for connection to the processor 4 is provided on an extending end of the scope cable 19.

The insertion section 9 is configured in a manner that a distal end portion 6, a bending portion 7, and a flexible tube portion 8 are continuously provided in a stated order from a distal end side. A distal end opening portion, an observation window, a plurality of illumination windows, an observation window cleaning port, and an observation object cleaning port which are well-known (not illustrated) are arranged for a distal end surface of the distal end portion 6.

An image pickup unit 30 which will be described below is installed on a back side of the observation window inside the distal end portion 6.

A distal end side of a light guide bundle which is not illustrated in the drawing is installed on a back side of the plurality of illumination windows. The light guide bundle is allowed to be inserted into an inside of the universal cord 11 from the insertion section 9 through the operation section 10. When the scope connector 18 is connected to the light source apparatus 3, illumination light from the light source apparatus 3 can be transmitted to the illumination windows.

The observation window cleaning port and the observation object cleaning port configure opening portions of two cleaning tubes (not illustrated) allowed to be inserted into the inside of the universal cord 11 from the distal end portion 6 through the operation section 10. These cleaning tubes are connected to a cleaning tank in which a cleaning fluid is stored and a compressor (none of which is illustrated) on a light source apparatus 3 side.

In the operation section 10, a forceps port 12 installed in a lateral portion on a lower section side, an operation section main body 13 configuring a grip portion of a midway section, a bending operation section 16 constituted by two bending operation knobs 14 and 15 provided on an upper section side, an air/water feeding control section 21, a suction control section 22, a switch section 23 configured by a plurality of switches to mainly operate an image pickup function, and an operation lever 24 for advance and retreat operations of a moving lens provided in the image pickup unit which will be described below are provided.

Note that the forceps port 12 of the operation section 10 configures an opening portion of a treatment instrument channel which is not illustrated in the drawing and which is arranged to be allowed to be mainly inserted into the insertion section 9 up to the distal end opening portion of the distal end portion 6.

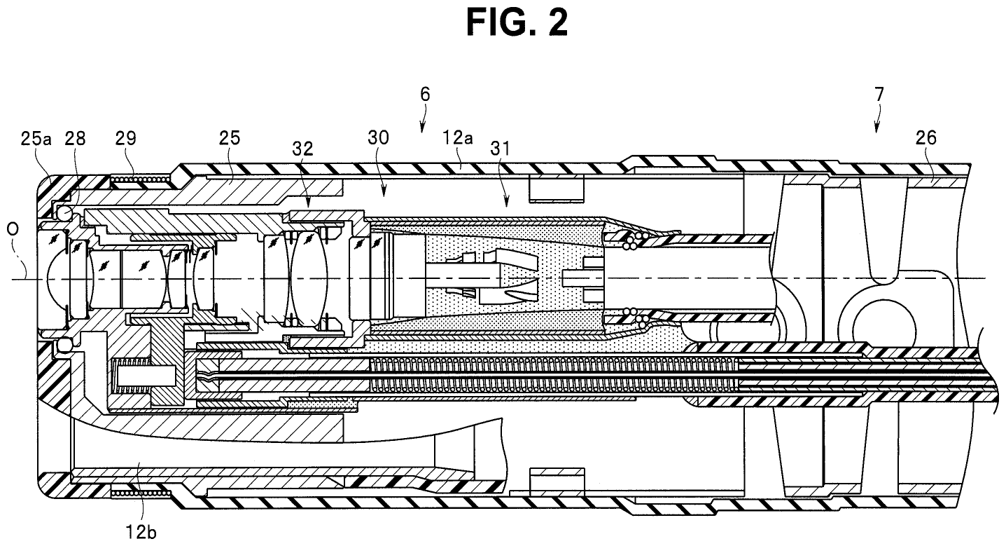

Next, a configuration of the distal end portion 6 of the endoscope 2 will mainly be described with reference to FIG. 2.

As illustrated in FIG. 2, the image pickup unit 30 is installed inside the distal end portion 6. The image pickup unit 30 is fitted to a hardened distal end rigid member 25 by insertion, and is fixed to the distal end rigid member 25 in a lateral direction by a set screw which is not illustrated in the drawing.

An O-shaped ring 28 for ensuring watertightness with the distal end rigid member 25 is installed in an outer circumferential portion on a distal end side of the image pickup unit 30. A distal end cover 25a configuring the distal end surface of the distal end portion 6 is fixed by bonding to a distal end side of the distal end rigid member 25.

Note that the distal end opening portion that is a hole portion formed in the distal end cover 25a configures an opening portion of a treatment instrument channel 12b in the distal end portion 6 as described above.

A plurality of bending pieces 26 configuring the bending portion 7 are continuously provided on a proximal end side of the distal end rigid member 25. Outer circumferences of the distal end rigid member 25 and the bending pieces 26 are integrally covered by an outer coat 12a made of rubber. A distal end outer circumferential portion of the outer coat 12a is fixed to the distal end rigid member 25 by a thread spool bonding portion 29.

Note that since members such as the cleaning tubes arranged in the distal end portion 6 and the light guide bundle for illumination are well-known components up to now, deceptions of those members will be omitted.

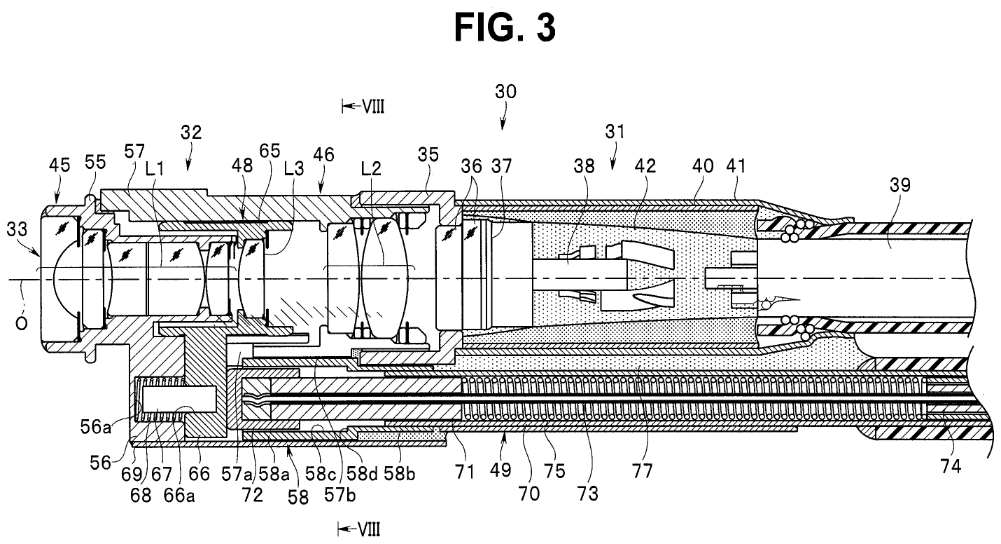

Next, a detailed configuration of the image pickup unit 30 will be described with reference to FIG. 3 to FIG. 8.

As illustrated in FIGS. 3, 4, 6, and 7, the image pickup unit 30 of the present embodiment is configured by including a solid state image pickup device unit 31, and an observation optical system unit 32 continuously provided on a distal end side of the solid state image pickup device unit 31.

The solid state image pickup device unit 31 includes a solid state image pickup device holding barrel 35. In the solid state image pickup device holding barrel 35, a front side of a solid state image pickup device chip 37 constituted by a CCD, a CMOS, or the like is held via an optical member 36 such as cover glass. A stacked substrate 38 is electrically connected to a back side of the solid state image pickup device chip 37 via an FPC or the like which is not illustrated in the drawing. Furthermore, a plurality of signal lines which are branched from an image pickup cable 39 serving as an internal component installed inside the insertion section 9 are connected to the stacked substrate 38. The image pickup cable 39 is connected to the electrical connector 20 through the inside of the endoscope 2. In this manner, the solid state image pickup device unit 31 can be electrically connected to the processor 4.

An enforcement barrel 40 is continuously provided in a proximal end outer circumferential portion of the solid state image pickup device holding barrel 35. A heat shrinkable tube 41 covering up to a distal end part of the image pickup cable 39 is provided in an outer circumference of the enforcement barrel 40. Note that a space formed by the enforcement barrel 40 and the heat shrinkable tube 41 from a proximal end portion of the solid state image pickup device holding barrel 35 is filled with a protective agent 42 such as a bonding agent so that the solid state image pickup device unit 31 is held in a watertight manner and is also protected.

The observation optical system unit 32 is configured by including an observation optical system 33 of a focus switching system which realizes a focusing function or a zooming function by causing an internal lens to move to advance or retreat in a direction of an optical axis O to change an optical characteristic (focal distance).

To describe more specifically, the observation optical system unit 32 is configured by including a front group lens unit 45 located on a distal end side, a rear group lens unit 46 continuously provided on a proximal end side of the front group lens unit 45, a moving lens unit 48 which is movable to advance or retreat in the optical axis O direction in the rear group lens unit 46, and an actuator 49 configured to cause the moving lens unit 48 to perform an advance or retreat operation.

The front group lens unit 45 is configured by including a front group lens barrel 55 that is a fixed barrel, and a front group lens L1 constituted by a plurality of fixed lenses held in the front group lens barrel 55.

A first stopper member 56 having an elongated flat plate shape, for example, is integrally formed in an outer circumferential portion on a proximal end side of the front group lens barrel 55. The first stopper member 56 is a member which defines an advance position in the optical axis O direction of the moving lens unit 48. A concave spring reception portion 56a is provided in a surface on a proximal end side in the optical axis O direction in the first stopper member 56.

The rear group lens unit 46 is configured by including a rear group lens barrel 57 that is a fixed barrel in which a distal end side is externally fitted to the front group lens barrel 55, and a rear group lens L2 constituted by a plurality of fixed lenses held on the optical axis O on a proximal end side of the rear group lens barrel 57.

The solid state image pickup device holding barrel 35 is externally fitted to the proximal end side of the rear group lens barrel 57. In this manner, the solid state image pickup device unit 31 and the observation optical system unit 32 are coupled to each other.

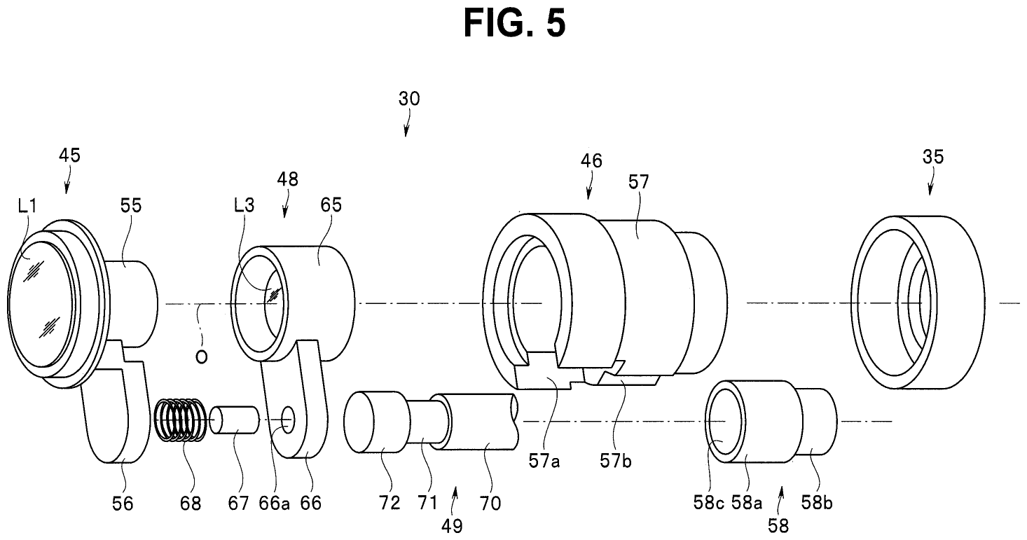

A slit-like guide groove 57a which penetrates through an inner circumferential side and an outer circumference side of the rear group lens barrel 57 is provided in the rear group lens barrel 57. A distal end side of the guide groove 57a is open in a distal end of the rear group lens barrel 57 (see FIG. 5). A proximal end side of the guide groove 57a extends halfway through the rear group lens barrel 57 in the optical axis O direction.

When the front group lens barrel 55 is mounted to the rear group lens barrel 57, the first stopper member 56 is inserted into the distal end side of the guide groove 57a. In this manner, in a state where the distal end side of the guide groove 57a is closed, the first stopper member 56 is arranged at a position protruding from an outer circumferential surface of the rear group lens barrel 57.

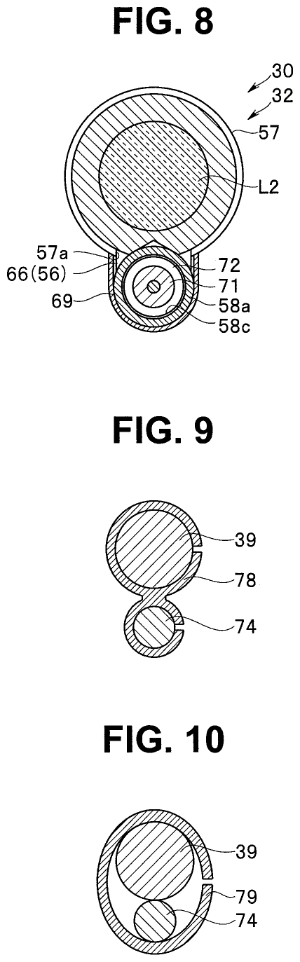

In the rear group lens barrel 57, a stopper holding portion 57b is provided in a region from a proximal end of the guide groove 57a to a mating portion of the rear group lens barrel 57 and the solid state image pickup device holding barrel 35. The stopper holding portion 57b is configured by a V-shaped groove which is linearly continuous to the guide groove 57a in the optical axis O direction, for example (see FIGS. 5 and 8).

Furthermore, a second stopper member 58 is fixed to the stopper holding portion 57b by bonding. The second stopper member 58 is a member which defines a retreat position in the optical axis O direction of the moving lens unit 48. The second stopper member 58 is being faced to the first stopper member 56 in the optical axis O direction by being fixed to the stopper holding portion 57b which is linearly continuous to the guide groove 57a.

According to the present embodiment, the second stopper member 58 is configured by a pipe member having a cylindrical shape, for example. More specifically, the second stopper member 58 is configured by including a first cylindrical portion 58a which abuts against the stopper holding portion 57b, and a second cylindrical portion 58b formed to have a diameter smaller than a diameter of the first cylindrical portion 58a to avoid interference with the solid state image pickup device holding barrel 35. An inside of the first cylindrical portion 58a and an inside of the second cylindrical portion 58b are set as an actuator holding hole 58c which penetrates in the optical axis O direction. Furthermore, a step portion 58d is formed in a boundary part between the first cylindrical portion 58a and the second cylindrical portion 58b in the actuator holding hole 58c.

The moving lens unit 48 is configured by including a moving lens barrel 65, and a moving lens L3 held in the moving lens barrel 65.

According to the present embodiment, the moving lens barrel 65 is, for example, a moving barrel which is movable to advance or retreat in the optical axis O direction inside the rear group lens barrel 57.

An operation member 66 having an elongated flat plate shape in which a shape in a plan view is similar to that of the first stopper member 56 is integrally formed in an outer circumferential portion of the moving lens barrel 65, for example. Before the front group lens barrel 55 is mounted to the rear group lens barrel 57, the operation member 66 is inserted into an inside of the guide groove 57a by the moving lens barrel 65 being inserted into the inside of the rear group lens barrel 57. In this manner, the operation member 66 protrudes from the outer circumferential surface of the rear group lens barrel 57. The operation member 66 faces the first stopper member 56 and the second stopper member 58. Furthermore, the operation member 66 is movable to advance or retreat in the optical axis O direction inside the guide groove 57a.

A bearing portion 66a which faces the spring reception portion 56a on a side opposite to the first stopper member 56 is provided in the operation member 66. A shaft member 67 which protrudes into an inside of the spring reception portion 56a is held in the bearing portion 66a. Furthermore, an outer circumference of the shaft member 67 is wrapped with a return spring 68 having one end side held in the inside of the spring reception portion 56a. The operation member 66 is biased to a proximal end side (side of the second stopper member 58) by the return spring 68.

Note that the first stopper member 56, the operation member 66, and the second stopper member 58 are covered by a cover 69 which closes the guide groove 57a in a watertight manner.

The actuator 49 includes a guide tube 70 serving as an actuator main body (movement section main body). The guide tube 70 has a distal end side held on a proximal end side of the second stopper member 58. In this manner, the second stopper member 58 also serves as a holding member which holds the distal end side of the guide tube 70. In other words, the distal end side of the guide tube 70 is held in the rear group lens barrel 57 without providing a dedicated holding member in the rear group lens barrel 57.

A push rod 71 which can freely move to advance or retreat inside the guide tube 70 is provided in the guide tube 70. A head member 72 serving as a motive power transmission member which abuts against the operation member 66 to be able to contact with or separate from the operation member 66 is fixed to a distal end of the push rod 71.

An outer diameter of the head member 72 is set at an outer diameter which allows sliding in an inner circumferential surface of the first cylindrical portion 58a configuring the second stopper member 58, and is also set at an outer diameter which allows engaging with the step portion 58d in which a proximal end of the head member is formed inside the second stopper member 58 when the push rod 71 retreats. In this manner, the step portion 58d realizes a function as a defining section which defines a stop position on a retreat side of the head member 72.

Herein, a length of the head member 72 is set at a length with which at least part of the head member 72 remains inside the actuator holding hole 58c in either state at the time of advance or the time of retreat of the push rod 71. In this manner, catch of the head member 72 with an opening portion on the distal end side of the second stopper member 58 is avoided, and a satisfactory advance and retreat movement is realized. In addition, the length of the head member 72 is set at a length with which the head member 72 is completely retracted into an inside of the actuator holding hole 58c when a proximal end of the head member 72 engages with the step portion 58d. In this manner, the retreat position of the moving lens unit 48 is defined by only the second stopper member 58 without being impacted by the head member 72.

A distal end side of a drive wire 73 allowed to be inserted into the inside of the guide tube 70 is continuously coupled to the push rod 71. A shape memory device 74 made of a shape memory alloy is continuously coupled to a proximal end side of the drive wire 73. Furthermore, inside the guide tube 70, an outer circumference side of the drive wire 73 is wrapped with a push spring 75 for biasing the push rod 71 toward a first stopper member 56 side with biasing force stronger than biasing force of the return spring 68.

The shape memory device 74 is set to contract at the time of heating and to expand at the time of cooling, for example. The shape memory device 74 is driven and controlled by a control unit 80 (see FIG. 1) provided in the operation section 10 according to an operation state on the operation lever 24.

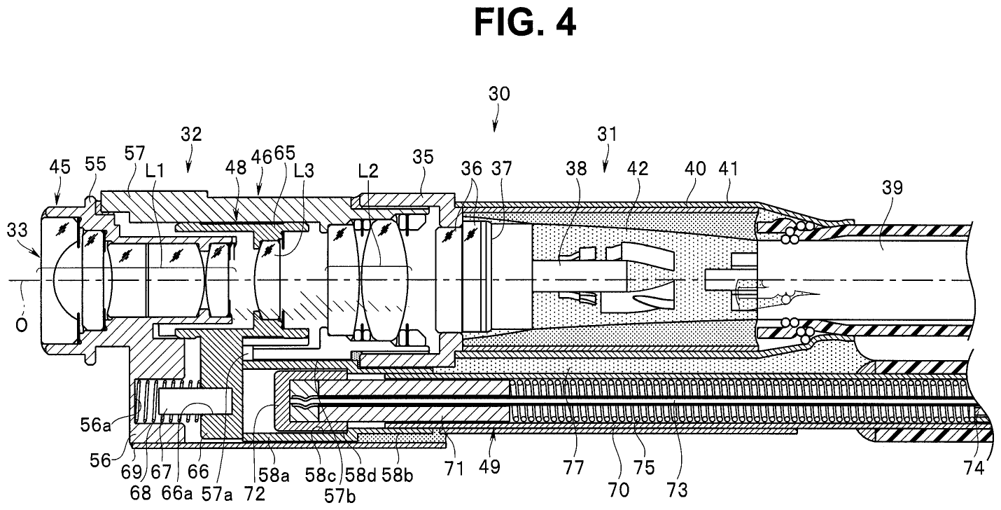

In other words, the control unit 80 turns on when a tilt operation is performed on the operation lever 24 toward a previously set retreat side. When power is on and the shape memory device 74 is heated, the shape memory device 74 contracts to cause the drive wire 73 to operate in a direction in which the biasing force of the push spring 75 is resisted (that is, in a direction on the proximal end side along the optical axis O). In this manner, the push rod 71 moves to retreat inside the actuator holding hole 58c. In this manner, the operation member 66 is biased by the return spring 68 to move to a position to abut against the second stopper member 58. Along with the movement of the operation member 66, the moving lens barrel 65 causes the moving lens L3 to move to a retreat position for realizing a previously set second focal distance (second optical characteristic) (see FIGS. 4 and 7).

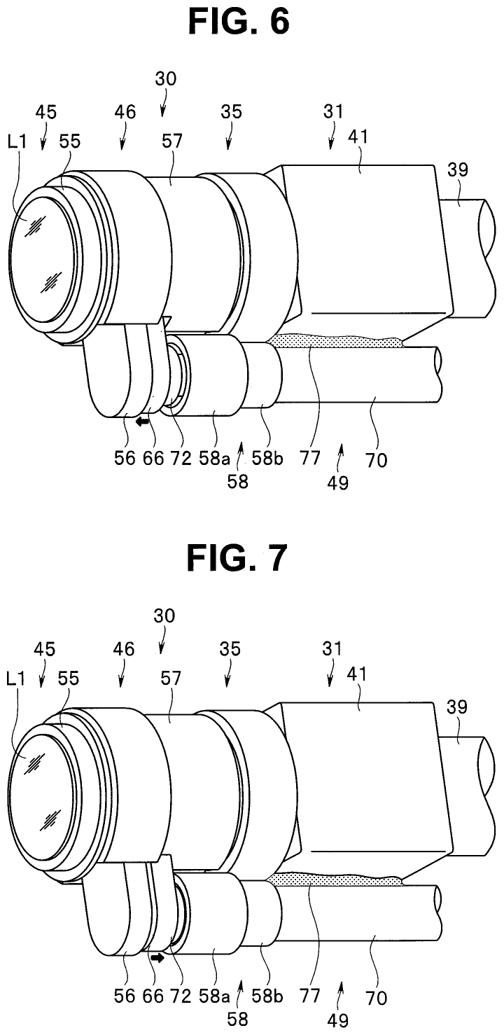

When a tilt operation is performed on the operation lever 24 toward a previously set advance side, the control unit 80 turns off the power to the shape memory device 74 to stop heat generation of the shape memory device 74. In this manner, the shape memory device 74 is expanded to cause the drive wire 73 to move in a direction to release the biasing force of the push spring 75 (that is, in a direction on the distal end side along the optical axis O). Due to the movement of the drive wire 73, the push rod 71 receives the biasing force of the push spring 75, and moves to advance inside the actuator holding hole 58c. By the movement of the push rod 71, the operation member 66 is pressed by the head member 72, and moves to advance to a position to abut against the first stopper member 56 against the biasing force of the return spring 68. In other words, the operation member 66 causes the moving lens L3 to move to an advance position for realizing a previously set first focal distance (first optical characteristic) (see FIGS. 3 and 6).

Herein, as illustrated in FIG. 9, for example, a proximal end side of the actuator 49 is integrally bound with an image pickup cable extending from the solid state image pickup device unit 31 inside the bending portion 7 or the like by binding instrument 78 serving as a binding member made of metal in a shape of figure of eight. In this manner, when the bending portion 7 is bent too, a load transmitted from the actuator 49 to the second stopper member 58 is reduced. Note that as illustrated in FIG. 10, for example, binding instrument 79 in an O shape can also be adopted as the binding member instead of the binding instrument 78 in the shape of figure of eight.

In the image pickup unit 30 thus configured, the advance position of the moving lens L3 for realizing the first focal distance is finely adjusted at the time of assembly of the observation optical system unit 32, for example. In other words, when the observation optical system unit 32 is assembled, in a state where the operation member 66 abuts against the first stopper member 56, the optical characteristic is checked based on an image or the like which is picked up by the solid state image pickup device chip 37, and a relative position between the front group lens barrel 55 and the rear group lens barrel 57 is positioned and fixed via a bonding agent.

The retreat position of the moving lens L3 for realizing the second focal distance is finely adjusted after the front group lens barrel 55 and the rear group lens barrel 57 are positioned and fixed, for example. In other words, in a state where the operation member 66 abuts against the second stopper member 58, for example, the optical characteristic is checked based on an image or the like which is picked up by the solid state image pickup device chip 37, and the second stopper member 58 is positioned by being caused to move in the optical axis O direction on the stopper holding portion 57b and fixed via a bonding agent 77. At this time, the bonding agent 77 is applied to not only an outer circumference of the first cylindrical portion 58a but also an outer circumference of the second cylindrical portion 58b. In other words, the second stopper member 58 is bonded and fixed to not only the stopper holding portion 57b of the rear group lens barrel 57 but also the solid state image pickup device holding barrel 35. At this time, an outer surface of the second stopper member 58 is treated to be rougher than the inner circumferential surface (the actuator holding hole 58c) of the second stopper member 58 by a shot blast treatment or the like, so that a bonding strength of the second stopper member 58 to the rear group lens barrel 57 and the solid state image pickup device holding barrel 35 can be further increased. In addition, when a space between the guide tube 70 of the actuator 49 and the heat shrinkable tube 41 is filled with the bonding agent 77 to fix the second stopper member 58, a fixation strength of the second stopper member 58 can be further improved.

Furthermore, to reduce power consumption at the time of retreat of the moving lens unit 48, calibration of a drive current is desirably performed for the actuator 49 at the time of manufacturing of the endoscope 2. In other words, at the time of assembly of the image pickup unit 30, for example, a current value necessary for causing the push rod 71 to retreat to a position where the proximal end of the head member 72 is caused to abut against the step portion 58d formed inside the actuator holding hole 58c is measured. A measured current value is stored in the control unit 80 (see FIG. 1) of the endoscope 2 as a reference current value, for example.

In this manner, for example, in a case where the moving lens unit 48 is moved to the retreat position, when a current supplied to the shape memory device 74 turns to the reference current value, the control unit 80 realizes a function of detecting that the head member 72 is locked to the step portion 58d and stopped. After this stop is detected, the control unit 80 maintains the current supplied to the shape memory device 74 at the reference current value. In this manner, a current supplied from the control unit 80 to the actuator 49 is controlled to be an appropriate value. Therefore, an excessive heat of the shape memory device 74 due to an excessive supply current is avoided. In addition, an application of an excessive tensile load to the second stopper member 58 due to an excessive contraction of the shape memory device 74 is avoided.

According to the above described embodiment, the image pickup unit 30 includes the observation optical system 33 which includes the rear group lens L2 and the moving lens L3, the rear group lens barrel 57 which holds the rear group lens L2, the moving lens barrel 65 which holds the moving lens L3 and can move to advance and retreat inside the rear group lens barrel 57 along the optical axis O of the observation optical system 33, the actuator 49 which includes the guide tube 70 and the head member 72 and transmits the motive power to the moving lens barrel 65 by the advance and retreat movement of the head member 72 with respect to the guide tube 70, and the second stopper member 58 which is positioned and fixed to the rear group lens barrel 57 and defines the retreat position in the optical axis O direction of the moving lens barrel 65, and the second stopper member 58 also serves as a holding member which holds the distal end side of the guide tube 70, so that durability of the second stopper member 58 is ensured, and also satisfactory workability at the time of assembly, the time of maintenance, or the like of the second stopper member 58 can be realized.

In other words, according to the present embodiment, a configuration is adopted in which the guide tube 70 is fixed to a proximal end side of the actuator holding hole 58c of the second stopper member 58 in which the head member 72 (and the push rod 71) moves to advance and retreat. Accordingly, a central axis of the guide tube 70 and a central axis of the actuator holding hole 58c can be matched on a same axis, and a load acting on the second stopper member 58 by the advance and retreat movement of the head member 72 can be reduced. In this way, when the load acting on the second stopper member 58 is reduced, the durability of the second stopper member 58 for itself can be improved, and also durability of a fixed portion of the second stopper member 58 to the rear group lens barrel 57 can be improved.

Since the second stopper member 58 is caused to also serve as the function of holding the guide tube 70 as described above, it is not necessary to provide a separate member which holds the guide tube 70 in the rear group lens barrel 57. Therefore, the second stopper member 58 can be formed to be long in the optical axis O direction, and the fixation strength of the second stopper member 58 to the rear group lens barrel 57 can be improved.

Since the second stopper member 58 is formed to be long in the optical axis O direction, gripping or the like of the second stopper member 58 using tweezers or the like at the time of assembly is facilitated, and assembly workability of the second stopper member can be improved. Therefore, the retreat position of the moving lens unit 48 which is defined by the second stopper member 58 can be set with a high precision.

In addition, at the time of maintenance or the like too, since the actuator 49 can be easily removed from the rear group lens barrel 57 by a removal operation of the second stopper member 58, maintainability of the image pickup unit 30 can also be improved.

In addition, since it is not necessary to provide a member which holds the distal end side of the guide tube 70 in the rear group lens barrel 57, a mating region for the solid state image pickup device unit 31 to be mated to the rear group lens barrel 57 can be expanded, and strength of the image pickup unit 30 itself can also be improved.

Herein, according to the above described embodiment, the processor 4 is configured by a well-known microcomputer including a CPU, a RAM, a ROM, a nonvolatile storage unit, and the like, and peripheral equipment of the microcomputer. A program executed by the CPU, fixed data such as a data table, and the like are stored in the ROM in advance. Note that all or part of functions of a processor may be configured by a logic circuit or an analog circuit, and processing of various programs may be realized by an electronic circuit such as an FPGA.

Note that the present invention is not limited to the respective embodiments described above. Various modifications and alterations can be made, and those are also within a technical scope of the present invention.

What is claimed is:

1. An endoscope comprising:
an observation optical system which is provided in a distal end portion of an insertion section inserted into an inside of a subject and which includes a fixed lens and a moving lens;
a fixed barrel which holds the fixed lens;
a moving barrel which holds the moving lens and which is movable to advance and retreat inside the fixed barrel along an optical axis of the observation optical system;
an actuator which includes a movement section main body and a motive power transmission member and which is configured to transmit motive power to the moving barrel by advance and retreat movement of the motive power transmission member with respect to the movement section main body; and
a stopper member which is positioned and fixed to the fixed barrel and which defines a retreat position in a direction of the optical axis of the moving barrel, wherein
the stopper member also serves as a holding member which holds a distal end side of the movement section main body.

2. The endoscope according to claim 1, wherein
the stopper member includes a defining section which defines a stop position on a retreat side of the motive power transmission member.

3. The endoscope according to claim 2, comprising:
a control unit configured to control the actuator, wherein
the control unit includes a function of detecting that the motive power transmission member stops at the stop position.

4. The endoscope according to claim 2, wherein
the defining section is a locking section to which the motive power transmission member is locked.

5. The endoscope according to claim 1, comprising:
a binding member which binds a proximal end side of the actuator to an internal component installed inside the insertion section.

6. An image pickup unit comprising:
an observation optical system which includes a fixed lens and a moving lens;
a fixed barrel which holds the fixed lens;
a moving barrel which holds the moving lens and which is movable to advance and retreat inside the fixed barrel along an optical axis of the observation optical system;
an actuator which includes a movement section main body and a motive power transmission member and which is configured to transmit motive power to the moving barrel by advance and retreat movement of the motive power transmission member with respect to the movement section main body; and
a stopper member which is positioned and fixed to the fixed barrel and which defines a retreat position in a direction of the optical axis of the moving barrel, wherein
the stopper member also serves as a holding member which holds a distal end side of the movement section main body.
